(19) United States
(12) Patent Application Publication
(10) Pub. No.: US 2007/0154895 A1
(43) Pub. Date: Jul. 5, 2007

(54) Multi-assay microfluidic chips

(75) Inventors: Michael Spaid, Mountain View, CA (US); Anne R. Kopf-Sill, Portola Valley, CA (US); Cheryl Cathey, Menlo Park, CA (US)

Correspondence Address: CALIPER LIFE SCIENCES, INC., 605 FAIRCHILD DRIVE, MOUNTAIN VIEW, CA, 94043-2234, US

(73) Assignee: Caliper Life Sciences, Inc., Mountain View, CA (US)

(21) Appl. No.: 11/323,992

(22) Filed: Dec. 30, 2005

(51) Int. Cl. C12Q 1/68; G01N 33/53; C12P 19/34; C12M 1/34

(52) U.S. Cl. 435/6; 435/7.1; 435/91.2; 435/287.2

(57) ABSTRACT

This invention provides systems and methods of running multiple different analyses on a microfluidic device. Results from such analyses can be useful in high throughput screening of samples or correlation of multiple as

### FIELD OF THE INVENTION

The present invention is in the field of microfluidic analyses. Multiple different assays of one or more test samples are run on the same microfluidic device. Methods of independently analyzing multiple samples with multiple different reagents on the same chip can be accomplished using separate sipper inputs from arrays of samples and reagents.

### BACKGROUND OF THE INVENTION

Automated analyses have been with us for some time. Many instruments are available to provide an analytical result for a sample at the push of a button. These instruments are typically integrated desktop devices where a computer controls actuators that route samples to contact reagents providing a specific signal at a detector. Such equipment often includes an autosampler carousel so that the operator can walk away while analyses continue for a series of samples.

Equipment also exists to run two or more different analyses on the same sample. For example, in the clinical environment there exist instruments that subaliquot a sample to multiple assay stations where assays, such as blood glucose, calcium, potassium, sodium, etc., are completed to provide a “panel” of results. In decades old technology, sequential multiple analyzer chemistry (SMAC) instruments receive a blood serum sample from a sample rack, dilute it in a stream of buffer solution, split the stream to 20 different analytical subsystems where reagents for 20 different chemistries are separately introduced to the split streams, and where detectors in each analytical subsystem detect a signal resulting from contact of the reagent with a sample analyte. Detector signals are communicated to a computer that can evaluate the quantity of analyte associated with each analytical subsystem. The several hundred pound SMAC analyzer used a sample size in the milliliter range and reagents by the liter to report 20 assay results on about 30 samples per hour.

Microfluidic chips offer many benefits over macro analyzers, such as the SMAC. Microfluidic chips have microscale channels to receive microliter or nanoliter-scale samples. Microliter-scale reagents, typically held in small wells on the chip, flow to contact a stream of the sample in a reaction channel before flowing on to a detector. Results are typically available in seconds. Each chip performs a single type of assay depending on the reagents loaded into the wells and the configuration of reaction channels and detectors.

It can be desirable to have results from multiple assays, in order to, e.g., enhance the precision of results, to screen multiple samples for rare analytes of interest, or to reduce false positive determinations in correlations of results to disease states. For example, many cancers are known to be associated with various tumor markers that could be screened for in clinical or blood banking sample libraries to provide earlier detection. High throughput would be valuable for multiple assays for statistical data establishing normal levels of markers, concern thresholds, and reliable correlations of disease states to marker patterns.

In view of the above, a need exists for a way to quickly run multiple different analyses on low volume samples. It would be desirable to have a way to quickly and inexpensively screen large numbers of samples with multiple analyses to develop more reliable marker thresholds, and marker pattern correlations so that more asymptomatic cancers can be confidently identified. The present invention provides these and other features that will be apparent upon review of the following.

### SUMMARY OF THE INVENTION

The present invention provides methods and systems for performing multiple different assays in a microfluidic device. In the methods, one or more samples are received into a microfluidic device to separately contact a plurality of reagents resulting in signals that can be detected and evaluated to determine the presence and/or quantity of two or more analytes of interest. The methods can be used to screen large numbers of samples for multiple analytes, or to correlate sample analyte patterns with certain disease states. In the systems for conducting multiple sample analyses, a microfluidic device with one or more reaction channels containing two or more reagents provide signals resulting from contact with samples. A detector detects the resultant signals for evaluation identifying or quantifying analytes of interest in the sample.

In one aspect, methods of multiple analyses, include receiving a sample into the microfluidic device, contacting the sample with a plurality of analytical reagents in the device, separately detecting signals resulting from two or more of the contacts between the reagents and the sample, and evaluating the resultant signals to determine the presence or quantity of one or more sample analytes to provide multiple analysis results for the sample. In another aspect, the methods of microfluidic analysis include consecutively receiving two or more samples into a microfluidic device, consecutively receiving two or more reagents into the microfluidic device, contacting the samples with the reagents in the device so that at least two of the samples contact at least one of the reagents and at least two of the reagents contact at least one of the samples, and detecting signals resulting from three or more of the contacts between samples and reagents. Detecting a resultant signal can include detecting the presence of an analyte in one or more samples, detecting the absence of detectable amounts of an analyte in one or more samples, and/or quantifying an analyte in one or more samples.

Samples received for analyses can be any testable materials, such as clinical materials, research materials, recombinant libraries, expression libraries, an infective agent, whole blood, serum, plasma, stool, urine, a vaginal secretion, cervical swab, ejaculatory fluid, synovial fluid, a biopsy, cerebrospinal fluid, amniotic fluid, or a forensic nucleic acid. Analytes of interest for detection by the multi-assay analyses methods can be any chemical or biological molecule, such as, e.g., a nucleic acid, a protein, a small molecule analyte, a drug, a lipid, a carbohydrate, an oncogene, a single nucleotide polymorphism (SNP), an antigen, an antibody, a methylated nucleic acid, a mutated nucleic acid, a single molecule, and/or the like. The samples can be received through a first pipettor tube and the reagents can be received through a second pipettor tube. The reagents can include any reagents useful in providing a detectable resultant signal on interaction with an analyte, e.g., a locus specific reagent, a PCR primer, a nucleic acid probe, a labeled ligand, a chromophore, an antibody, a fluorophore, an enzyme, a fluorescent resonant energy transfer (FRET) probe, a molecular beacon, a radionuclide, and/or the like.

The samples and reagents can be received into the microfluidic device in any suitable combination or order. For example, two or more samples can be consecutively received into the device while one reagent is continuously received. In many embodiments samples are split to flow into a plurality of reaction channels in the device to separately contact the each of the plurality of reagents. In a preferred embodiment, the higher dispersion specimens (member) of either the reagents or the samples is continuously received while two or more of the lower dispersion member are consecutively received. Contacting in the microfluidic device typically takes place in microscale channels (microchannels) of the device.

In particularly preferred embodiments of the multi-assay methods, at least one of the assays run on the microfluidic device is a continuous flow polymerase chain reaction (PCR) a single analyte molecule detection, a mutant DNA assay, a methylated DNA assay, or an assay for detection of an antigen. In one embodiment of the invention the multi-assay method includes assays for both a nucleic acid and a peptide on the same microfluidic device.

Multiple assay results can be evaluated to determine the presence or quantity of analytes of interest. The presence, quantity, or pattern of analytes can be correlated to disease states associated with the sample. For example, multiple analysis results can be used to screen patients for disease states or pathogens. Assay results can be correlated with a disease state, such as a cancer.

Systems for conducting multiple sample analyses have the flexibility of design to run one or more samples against two or more reagents in one or more reaction channels providing a variety of resultant signal combinations. Multi-assay systems can include, e.g., a microfluidic device with one or more reaction channels, two or more reagents flowing to contact a sample in the one or more reaction channels, and one or more detectors configured to detect one or more signals resulting from two or more of the contacts between the reagents and the sample. The resultant signals can be evaluated to determine the presence or quantify of two or more analytes in the sample. The systems can be useful for analyzing a matrix of sample/reagent combinations, e.g., with a first pipettor tube configured to sequentially receive two or more samples, a second pipettor tube configured to sequentially receive two or more reagents, a microfluidic device with a reaction channel in fluid connection with the first pipettor tube and with the second pipettor tube so that the samples and reagents can come into contact in the channel, and a detector configured to detect one or more signals resulting from the contact of the samples with the reagents providing a matrix sample to reagent analytical results.

The pipettors can be part of one or more specimen stations to facilitate receipt of the samples or reagents. Typical specimen stations include, e.g., a carousel style autosampler, a multiwell plate on a X-Y translatable base, or a microarray chip sampled by a translatable pipettor.

Samples and reagents in the systems can be the same as described above for the methods of the invention. For example, samples can include: a nucleic acid with single nucleotide polymorphism (SNP), a cancer associated nucleic acid, a nucleic acid from an infective agent, whole blood, serum, plasma, stool, urine, a vaginal secretion, cervical swab, ejaculatory fluid, synovial fluid, a biopsy, cerebrospinal fluid, amniotic fluid, or a forensic nucleic acid. The samples can include analytes of interest, such as a nucleic acid, a protein, a small molecule analyte, a drug, a lipid, a carbohydrate, an oncogene, a single nucleotide polymorphism (SNP), an antigen, an antibody, a single molecule, a DNA mutation, a methylated DNA, an antigen and/or the like. In one embodiment, the analytes assayed in the system include both a protein and a nucleic acid. The reagents can include, e.g., a locus specific reagent, a PCR primer, a nucleic acid probe, a labeled ligand, a chromophore, an antibody, a fluorophore, an enzyme, a fluorescent resonant energy transfer (FRET) probe, a molecular beacon, a radionuclide, and/or the like.

The microfluidic device preferably includes multiple reaction channels. The reaction channels can be thermocycler amplification channels, chromatographic channels, incubation channels, affinity capture channels, etc. The reaction channels can also include detection regions or lead into detection regions for detection of resultant signals.

Resultant signals can be detected by any appropriate detectors. The detector can separately detect signals from two or more of the reaction channels in series or in parallel. The resultant signals providing information about analytes in the samples can be, e.g., detectable signals from reagents that have reacted sample analytes, a lack of a detectable signal, and/or a signal amplitude related to a quantity of a sample analyte. The detector can be, e.g., a fluorometer, a charge coupled device, a laser, an enzyme, an enzyme substrate, a photo multiplier tube, a spectrophotometer, scanning detector, microscope, a galvo-scanner, and/or the like.

The multi-assay systems can include a computer. The computer can be in functional communication with the one or more detectors to receive detector signals. The computer can include a system software program useful for: evaluation of the detector signals for the presence of one or more of the analytes, evaluation of the detector signals to quantify one or more of the analytes, screening the sample for the presence of one or more pathogens, correlating the presence or quantity of two or more analytes to a disease state, controlling system flows, controlling system voltages, and the like.

### Definitions

Unless otherwise defined herein or below in the remainder of the specification, all technical and scientific terms used herein have meanings commonly understood by those of ordinary skill in the art to which the present invention belongs.

Before describing the present invention in detail, it is to be understood that this invention is not limited to particular methods or devices, which can, of course, vary. It is also to be understood that the terminology used herein is for the purpose of describing particular embodiments only, and is not intended to be limiting.

As used in this specification and the appended claims, the singular forms “a”, “an” and “the” include plural referents unless the content clearly dictates otherwise. Thus, for example, reference to “a detector” can include a combination of two or more detectors; reference to “nuclides” can include individual nuclides, and the like.

Unless defined otherwise, all technical and scientific terms used herein have the same meaning as commonly understood by one of ordinary skill in the art to which the invention pertains. Although many methods and materials similar, modified, or equivalent to those described herein can be used in the practice of the present invention without undue experimentation, the preferred materials and methods are described herein. In describing and claiming the present invention, the following terminology will be used in accordance with the definitions set out below.

The term “reagent”, as used herein, refers to one or more moieties that interact with sample analytes of the invention to provide a resultant signal. A reagent can be in chemical in solution, suspension, and/or bound to a surface. A reagent can be a selective (e.g., chromatographic) media in a reaction channel of the multi-assay microfluidic systems of the invention. A “reagent” can be, e.g., two or more molecules and/or media that work together to provide the detectable resultant signal. “Different reagents”, as used herein include two or more reagents that each interact with a different analytes to provide a resultant signal, or each provide resultant signals based on different characteristics (e.g., size, activity, ligand binding sites, etc.) of the same analyte.

As used herein, the term “resultant signal” refers to a signal resulting from contact of a sample with a reagent. The resultant signal can, e.g., can be a detectable property of the sample analyte of interest (e.g., absorbance wavelength) or a property of the reagent (e.g., a probe label). A resultant signal can be a lack of a detectable signal, e.g., at a time when a signal would have been expected for a positive control sample.

### BRIEF DESCRIPTION OF THE DRAWINGS

FIG. 1 is a schematic diagram of an exemplary system of the invention having multiple specimen stations to provide a variety of sample/reagent combinations to the microfluidic device of the system.

FIG. 2 is a schematic diagram of a system having reaction channels configured to run three different analyses on the same microfluidic device.

FIG. 3 is a schematic diagram of a system configured for the capability of running a series of multiple samples with a series of multiple reagents.

FIGS. 4A to 4C are schematic diagrams of exemplary layouts for pipettor tubes and microfluidic device channels.

FIG. 5 is a schematic diagram of an exemplary system for running multiple different assays on a microfluidic device.

### DETAILED DESCRIPTION

The present invention provides systems and methods to run multiple assays on samples in a microfluidic device, e.g., for high throughput screening or to generate data for dependable correlations of a sample to a disease state. Systems of the invention allow detection or quantitation of one or more sample analytes with more than one analytical method on the same microfluidic chip. Systems can expedite handling of multiple samples and multiple reagents by feeding specimens to the microchip through two or more pipettor tubes. Methods of the invention include concurrently running multiple different analyses on a microfluidic chip and evaluating detector signals to determine the presence or quantity of sample analytes. The methods can include consecutively receiving two or more samples and two or more reagents into a microfluidic device through two or more pipettor tubes, bringing the samples into with the reagents and detecting signals from the contacts to determine the presence or quantity of two or more analytes in the samples.

Methods and systems of the invention can provide high throughput accumulation of analyte identity and quantity data for one or more samples. Large numbers of analyses can be rapidly performed using small sample and reagent volumes. Multiple different assays can be run by receiving a series of different reagents into the microfluidic device or multiple different assays can be run in parallel on the same chip. A large number of samples can be screened for an analyte and/or a sample can be screened for a large number of analytes. The diverse assay data provided for a sample can be correlated to disease states with accuracy unobtainable from single assay methods.

Multiple Assay Microfluidic Systems

Systems of the invention include microfluidic devices with multiple reaction channels to analyze two or more samples at once and/or to run two or more analyses at once. For example, a sample can be received into the device and split to two or more reaction channels where sample analytes come in contact with separate analytical reagents resulting in detectable signals that can be interpreted to determine the presence and/or amount of two or more analytes in the sample. Systems of the invention can include specimen sampling systems capable of selectively delivering samples and reagents to the device at the same time.

The systems can be configured to run multiple analyses on multiple samples. For example, the systems can receive for analysis: a continuous flow of a single sample, a series of different samples, multiple different samples at once, and/or a series of multiple different samples. The systems can provide sample contact with, e.g.: a single reagent, a series of different reagents, multiple different reagents at once, and/or a series of multiple different reagents. Systems of the invention can be configured, e.g., with any sample reception/reagent contact combination appropriate to the data acquisition, screening, and/or correlation requirements of the user.

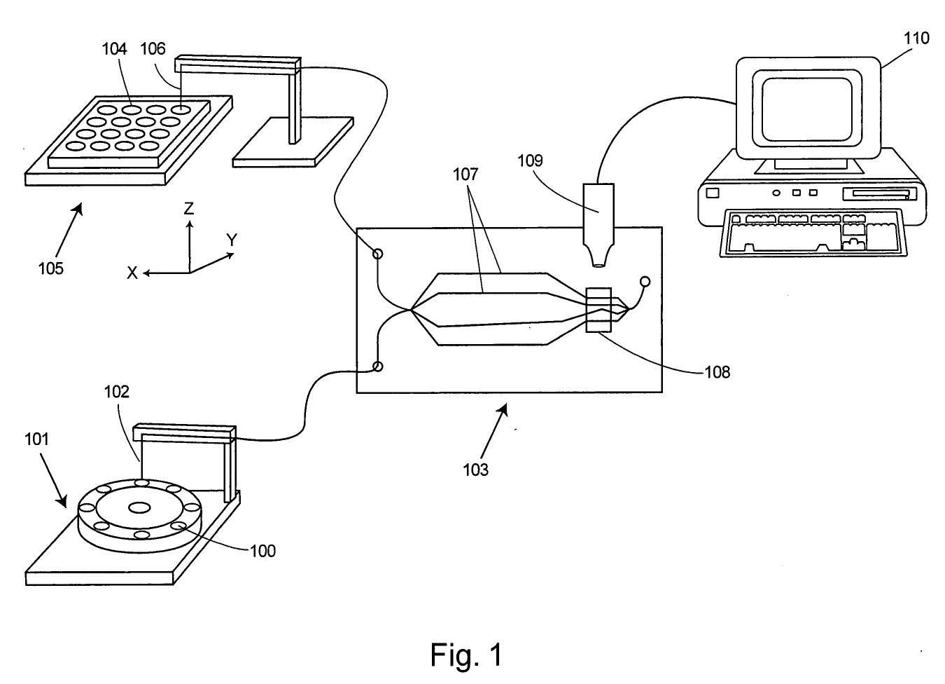

A typical multiple assay system of the invention is shown schematically in FIG. 1. The system allows multiple samples to sequentially contact multiple reagents for replicate analyses in multiple parallel reaction channels followed by separate signal detection for each replicate assay. In the system, reagents 100 in reagent specimen station 101 are sampled through reagent pipettor tube 102 to flow into microfluidic device 103. Samples 104 in sample specimen station 105 are sampled through sample pipettor tube 106 to be received into the microfluidic device and contact the reagents in reaction channels 107. After contact between the samples and reagents in the reaction channels, resultant signals from the reaction mixtures are independently detected as they flow through multiple flow cells in detection region 108 monitored by detector 109. Detector signals can be communicated to computer 110 for interpretations and correlations.

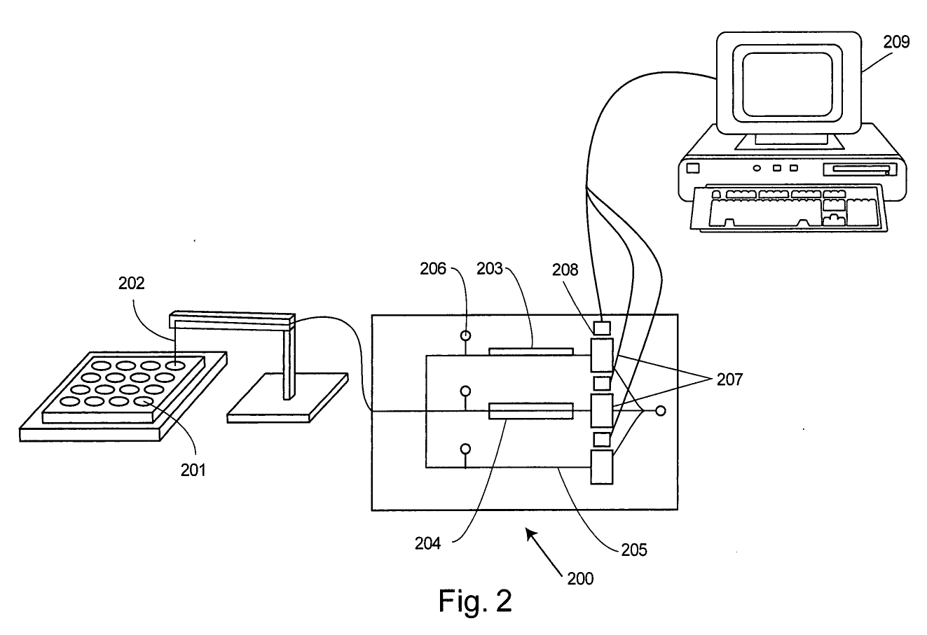

In another embodiment, as shown schematically in FIG. 2, the system includes multiple reaction channels in the microfluidic device for different analyses to be run at the same time. The microfluidic device 200 includes three reaction channels, each configured for a different type of analysis. Samples 201 are sampled through sample pipettor tube 202 and flow into the device to split in channels leading to three different reaction channels: capillary electrophoresis channel 203, thermocycling amplification channel 204, and ELISA incubation channel 205. Assay solutions or reagents in wells 206 can flow to admix with samples, as necessary. Each reaction channel flows into a separate detection region 207 for detection of signals resultant from each assay reaction by detectors 208. Detector signals are communicated to computer 209 for interpretation and correlation.

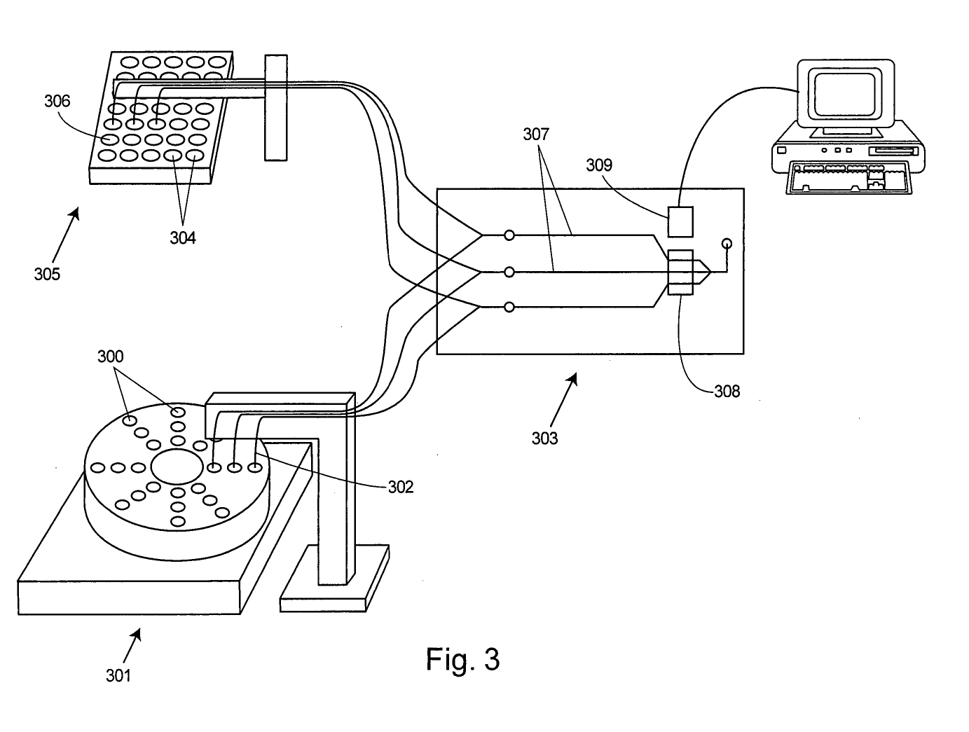

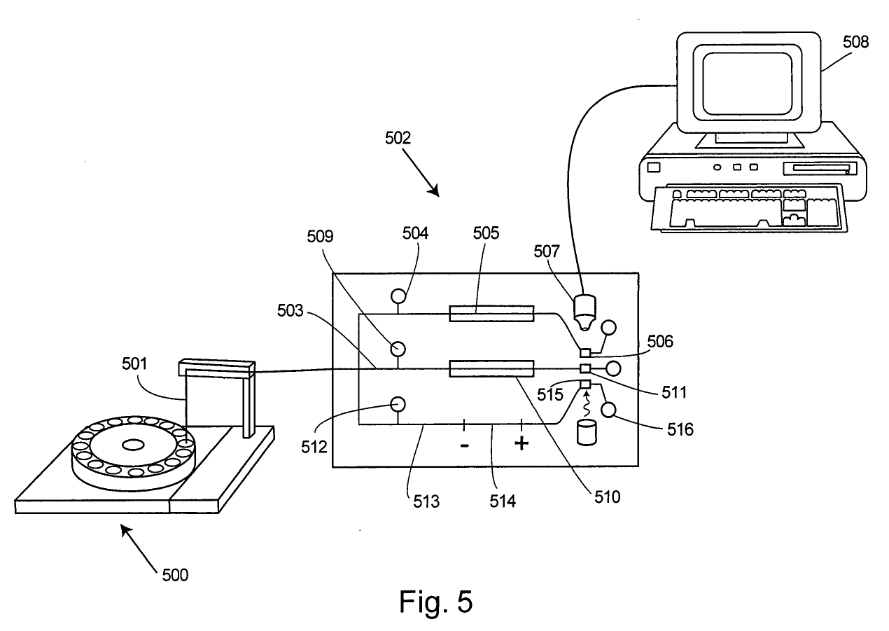

The microfluidic devices, e.g., in cooperation with pipettors and specimen stations, can be configured to provide the logical combinations and permutations of sampling and contact options. For example, multiple analyses can be provided by: 1) running a single sample with a series of reagents (e.g., see FIG. 2), 2) running a single sample with multiple reagents in parallel, 3) running a single sample with a series of multiple reagents in parallel, 4) running a series of samples with a series of reagents (e.g., see FIG. 1), 5) running a series of samples each with multiple reagents in parallel (see, e.g., FIG. 5), 6) running a series of samples with a series of multiple reagents in parallel, 7) running multiple parallel samples with the same reagent, 8) running multiple parallel samples with a series of reagents, 9) running multiple parallel samples with multiple parallel reagents (e.g., see FIG. 3), 10) running multiple parallel samples with a series of multiple parallel reagents, 11) running a series of multiple parallel samples with the same reagent, 12) running a series of multiple parallel samples with a series of reagents, 13) running a series of multiple parallel samples with multiple parallel reagents, and/or 14) running a series of multiple parallel samples with a series of multiple parallel reagents. Running a series of samples or reagents can generally be accomplished, e.g., by sampling a series from a specimen station through a pipettor tube. Running multiple parallel samples or reagents can generally be accomplished, e.g., by sampling in parallel through multiple pipettor tubes into multiple reaction channels of the device, or by time-sharing one or more pipettor tubes to load the multiple reaction channels.

Running a series of multiple parallel samples with a series of multiple parallel reagents can be accomplished as follows. Reagents 300 in reagent specimen station 301 can be sampled by multiple reagent pipettor tubes 302 to provide a first multiple set of reagents to be received by microfluidic device 303, as shown in FIG. 3. Samples 304 in sample specimen station 305 can be sampled by multiple sample pipettor tubes 306 to provide a first multiple set of samples to be received by the microfluidic device and to contact the reagents in multiple reaction channels 307. The sample/reagent mixtures can flow to detection region 308 for detection of resultant signals by detector 309. After an adequate amount of the first multiple set of reagents and samples has been sampled at the stations, a second in a series of multiple sets of reagents and samples can be sampled, flow to contact, through to the reaction channels and the detection region to provide analysis of a series of multiple samples with a series of multiple reagents. One skilled in the art can appreciate how other multiple analyses, e.g., described in the paragraph above, can be run using systems of the invention.

Multiple sampling can be accomplished in a variety of ways. Optionally, multiple samples or reagents can be sampled using multiple specimen stations, each with individual pipettor tubes. Optionally, multiple samples and multiple reagents can be sampled using a single specimen station having both sample and reagent pipettor tubes. In some embodiments, a single pipettor tube can sample multiple samples and/or reagents for loading into devices for contacts in multiple reaction channels. Such loaded reaction channels can flow with synchronized or staggered reaction timing toward the detection region.

Microfluidic Devices

Microfluidic devices of the systems have one or more microscale channels and are capable of running two or more different analyses. The devices can be configured to receive multiple samples and/or provide for contacts with multiple reagents. Samples and/or reagents can flow into the device through one or more pipettor tubes, or optionally from wells on the device. Multiple contacts can take place sequentially in a reaction channel and/or contemporaneously in multiple parallel reaction channels. Detection of signals resulting from contact between samples and reagents can take place sequentially, and/or multiple signals can be detected at once. Such diverse capabilities can be designed into the devices of the invention, e.g., by selecting a suitable combination of specimen samplers to pipettor tubes, single or parallel reaction channels, and single or parallel detector systems.

Microfluidic devices can have channels, e.g., embedded on the surface of a substrate by mold injection, photolithography, etching, laser ablation, and the like. At least one channel of the device can have a microscale dimension, such as, e.g., a depth or width ranging from about 500 μm to about 0.1 μm, from about 100 μm to about 1 μm, or about 10 μm. Fluids can flow in the channels, e.g., by electroosmotic flow (EOF), capillary action (surface tension), hydraulic or pneumatic pressure differentials, gravity, and/or the like. Channels can terminate, e.g., in pipettor tubes, in wells of solutions, and/or at intersections with other channels or chambers. Channels can have electrical contacts, e.g., at each end to provide electric fields and/or electric currents to separate analytes or to induce EOF. Detectors can be functionally associated with channels to monitor parameters of interest, such as, e.g., voltages, conductivity, resistance, capacitance, electric currents, refractivity, light absorbance, fluorescence, pressures, flow rates, and/or the like. Microfluidic chips can have functional information communication and utility connections to supporting instrumentation, such as electric power connections, vacuum sources, pneumatic pressure sources, hydraulic pressure sources, analog and digital communication lines, optic fibers, etc.

The microfluidic devices can have sample channels and reagent channels in fluid contact with reaction channels so that samples and reagents can come into contact to form a reaction mixture that can generate a specific signal. Sample channels and reagent channels can be in fluid contact with the samples or reagents in wells of the microfluidic device, or preferably in fluid contact with pipettor tubes associated with specimen stations to potentially receive multiple specimens in series (consecutively) or in parallel (at once).

Reaction channels in the microfluidic devices are channels where samples (or sample constituents, such as analytes of interest) come into contact with assay specific reagents. Reaction channels can be configured to provide conditions necessary to provide a detectable signal resulting from the contact. For example, reaction channels can receive forces to induce flows, have controlled temperatures, have sufficient lengths to provide adequate incubation times, have solid supports to hold or capture reaction constituents, hold selective media, and/or the like. The devices can have a single reaction channel or multiple reaction channels.

Reaction channels can be, e.g., thermocycler amplification channels that cycle through a programmable temperature profile a number of times while the reaction mixture is present in the channel. Amplification reactions in thermocycling channels are typically polymerase chain reactions (PCR) to amplify rare or dilute nucleic acid sequences from a sample so they can be detected. A number of high throughput approaches to performing PCR and other amplification reactions have been developed, e.g., involving amplification reactions in microfluidic devices, as well as methods for detecting and analyzing amplified nucleic acids in or on the devices. Details regarding such technology is found, e.g., in the technical and patent literature, e.g., Kopp et al. (1998) “Chemical Amplification: Continuous Flow PCR on a Chip” Science, 280 (5366):1046; U.S. Pat. No. 6,444,461 to Knapp, et al. (Sep. 3, 2002) MICROFLUIDIC DEVICES AND METHODS FOR SEPARATION; U.S. Pat. No. 6,406,893 to Knapp, et al. (Jun. 18, 2002) MICROFLUIDIC METHODS FOR NON-THERMAL NUCLEIC ACID MANIPULATIONS; U.S. Pat. No. 6,391,622 to Knapp, et al. (May 21, 2002) CLOSED-LOOP BIOCHEMICAL ANALYZERS; U.S. Pat. No. 6,303,343 to Kopf-Sill (Oc. 16, 2001) INEFFICIENT FAST PCR; U.S. Pat. No. 6,171,850 to Nagle, et al. (Jan. 9, 2001) INTEGRATED DEVICES AND SYSTEMS FOR PERFORMING TEMPERATURE CONTROLLED REACTIONS AND ANALYSES; U.S. Pat. No. 5,939,291 to Loewy, et al. (Aug. 17, 1999) MICROFLUIDIC METHOD FOR NUCLEIC ACID AMPLIFICATION; U.S. Pat. No. 5,955,029 to Wilding, et al. (Sep. 21, 1999) MESOSCALE POLYNUCLEOTIDE AMPLIFICATION DEVICE AND METHOD; U.S. Pat. No. 5,965,410 to Chow, et al. (Oct. 12, 1999) ELECTRICAL CURRENT FOR CONTROLLING FLUID PARAMETERS IN MICROCHANNELS; Service (1998) “Microchips Arrays Put DNA on the Spot” Science 282:396-399), Zhang et al. (1999) “Automated and Integrated System for High-Throughput DNA Genotyping Directly from Blood” Anal. Chem. 71:1138-1145, and many others.

In some cases, reaction channels can merely act as incubators and/or mixers with conditions of flow time and temperature adequate, e.g., for an analyte to specifically react with a chromophore. For example, many proteins, ions, or nucleic acids can interact with certain dyes to produce detectable signals of color, light absorbance or fluorescent emissions while flowing through a reaction channel.

In other cases, reaction channels can include reagents in the form of selective media. Selective media can be those known in the art, such as, e.g., size selective media (e.g., size exclusion media or electrophoresis gels), ampholyte buffers used in isoelectric focusing (IEF) techniques, ion exchange media, affinity media (e.g., lectin resins, antibodies attached to solid supports, metal ion resins, etc.), hydrophobic interaction resins, chelator resins, and/or the like. The selective media can be considered “reagents” of the invention when sample contact results in resolution or capture of analytes allowing detection of a specific resultant signal. For example, contact of a sample with a size exclusion media reagent can resolve a protein analyte of interest from other sample constituents so that a 280 nm absorbance signal after a predetermined retention time can be interpreted to determine the presence or quantity of the protein in the sample.

Microfluidic devices can have detection regions that can be monitored by detectors of the systems to detect the signals resulting from contact of samples with reagents. The detection regions can be one or more channel segments or chambers in functional contact with sensors. For example, detector regions can incorporate sensors such as pH electrodes conductivity meter electrodes. Detection regions can comprise one or more channels or chambers transparent to certain light wavelengths so that light signals, such as, e.g., absorbance, fluorescent emissions, chemoluminescence, and the like, can be detected.

Samples and Reagents

Samples and reagents can be combined in reaction channels to ultimately produce a detectable signal due to a reaction or interaction between the reagent and a sample analyte.

Samples can be any material potentially containing an analyte of interest. The samples can be clinical samples, members of a molecular library, research samples, process samples, and the like. Analytes of interest can include, e.g., a nucleic acid, a protein, a small molecule analyte, a drug, a lipid, a carbohydrate, an oncogene, a single nucleotide polymorphism (SNP), an antigen, an antibody, a single molecule, and/or the like. In some embodiments, a sample of interest can include, or potentially include, two or more analytes of interest.

Reagents can be any composition capable of interacting with an analyte of interest to generate a detectable resultant signal. Reagents are typically one or more molecules in a solution that can flow into contact with a sample in a reaction channel to specifically interact with an analyte to produce a specific detectable signal. For example, reagents can be a chromophore that reacts with the analyte with some specificity to provide a changed optical signal. Reagents in the systems can also include molecules attached to media (e.g., a gel or solid support) and capable of interacting with analytes of a sample to allow detection in the detection region. For example, the reagent can be an affinity molecule on a solid support that captures the analyte for presentation to the detector. More than one reagent can be involved in generating a signal. For example, in an ELISA type sandwich assay, one reagent can be a capture antibody, another reagent can be an antibody against the analyte and linked to a chromogenic enzyme, and a third reagent can be the chromophore substrate to the enzyme. Typical reagents on the systems of the invention include, e.g., a locus specific reagent, a PCR primer, a nucleic acid probe, a labeled ligand, a chromophore, an antibody, a fluorophore, an enzyme, a fluorescent resonant energy transfer (FRET) probe, a molecular beacon, a radionuclide, and/or the like.

Specimen Stations

It is an aspect of the invention that the microfluidic device can sequentially receive two or more samples from one pipettor and sequentially receive two or more reagents from a second pipettor to provide a matrix of sample and reagent combinations for analysis in the systems. Sampling stations can reliably bring samples and/or reagents (specimens) into functional contact with pipettors so that desired sample/reagent contacts can occur in the devices. Samples and/or reagents can be received into microfluidic devices of the systems, e.g., by pipetting them from aliquots present in specimen stations. The stations can have any design suitable for holding and presenting the specimens for sampling by a pipettor tube.

Specimen stations can be, e.g., autosamplers, such as sample carousels holding multiple specimens in a circular tray that can be rotated sequentially or randomly to align specimen containers with one or more pipettors. The pipettors can be on actuated arms that can dip the pipettor tube into the specimen for sampling.

Specimen stations can be configured to hold one or more microtiter plates of specimens. The station can translate the plates with an X-Y plotting motion to position any of the plate wells under a pipettor tube. Alternately, the pipettor tube can be on an arm capable of an X-Y plotting motion to position the pipettor over intended specimen wells. The arm and/or plate can move in a vertical (Z) direction to dip the pipettor tip into the well for sampling.

In some embodiments, one of more of the pipettors are sipper tubes projecting directly from the microfluidic device so that proper relative positioning of the device and a specimen station can place the tip of the pipettor into a desired specimen for sampling and receipt into the device. Such a design can minimize sampling size and the time for specimens to reach the reaction channels.

In many embodiments of the systems, the samples or reagents are of very small volume, e.g., as is typical of many molecular libraries. Sampling from such libraries, e.g., on microwell plates or microarray slides, is typically accomplished with robotic systems that precisely position the pipettor tip in the micro specimen. In embodiments where the library members are retained in dehydrated form, it can be convenient to sample by ejecting a small amount of solvent from the pipettor to dissolve the specimen for receipt into the device.

Specimen stations can be designed to allow synchronous or independent specimen sampling, according to an intended contact sequence. For example, associated samples and reagents in a specimen station can be arranged so that a single sampling arm with both a reagent pipettor tube and a sample pipettor tube to receive both samples and their associated reagents at once. In another example, separate sampling arms in one station, or separate sample and reagent specimen stations, can be provided so that, e.g., a sample can be continuously sampled through one pipettor tube while a series of different reagents are sampled from another pipette tube. Such an arrangement can allow, e.g., independent sampling of reagent and/or sample series, or serial loading of multiple reaction channels with different combinations of reagents and samples.

Detectors of Signals

Detectors in the multi-assay systems can be capable of detecting a signal that results from an interaction of an analyte of interest with a reagent. Detectors can be located in microfluidic device, or proximate to the device, in an orientation to receive signals resulting from the sample contact with the reagent. Detectors are typically disposed to detect signals coming from a detection region of the device. Detectors can detect a signal from a reagent that has reacted with a sample analyte, the absence of a detectable signal (interpretable, e.g., as the absence of sample analyte at a level adequate to generate a signal above the sensitivity of the detector), a signal amplitude related to a quantity of a sample analyte, and/or the like.

Detectors can be, e.g., hardware that works to detect signals of reagent/sample interactions. Detectors can include, e.g., a fluorometer, a charge coupled device, a laser, a photo multiplier tube, a spectrophotometer, scanning detector, microscope, or a galvo-scanner. Signals detected from interactions of reagents and samples can be, e.g., absorbance of light wavelengths, light emissions, radioactivity, conductivity, refraction of light, etc. The character of signals, such as, e.g., the amplitude, frequency, duration, counts, and the like, can be detected.

Detectors can detect signals from detector regions described by physical dimensions, such as a point, a line, a surface, or a volume from which a signal can emanate. In many embodiments, the detector monitors a detection region that is essentially the point along a channel where a reaction mixture flows out from a reaction channel. In other embodiments, the detector can scan a detection region along the length of a channel while the reaction mixture is flowing or stopped. In still other embodiments, the detector can scan an image of a surface or volume for signals resulting from interactions of reagents and samples. For example, a detector (e.g., a charge coupled device) can contemporaneously image multiple parallel channels carrying reaction mixtures from multiple analyses to detect results of several different assays at once.

The detectors can transmit detector signals that express characteristics of resultant signals received. For example, the detector can be in communication with an output device, such as an analog or digital gage, that displays a value proportional to a resultant signal intensity. The detector can be in communication with a computer through a data transmission line to transmit analog or digital detector signals for display, storage, evaluation, correlation, and the like.

Computers

Computers in multi-assay systems of the invention can control system processes and receive signals for interpretation. For example, the computer can control a robotic sub-system that retrieves samples or analytes from storage as needed for the intended assay runs. The computer can control specimen stations to designate the order of sipping samples and reagents for receipt into the microfluidic device. Pressure differentials and electric potentials can be applied to Microfluidic devices by the computer through computer interfaces known in the art. The computer can be a separate sub-system, it can be housed as an integrated part of a multi-assay instrument, or dispersed as separate computers in modular subsystems.

The computer system for controlling processes and interpreting detector signals can be any known in the art. The computer can control power circuits, control mechanical actuators, receive the information through communication lines, store information, interpret detector signals, make correlations, etc. Systems in the present invention can include, e.g., a digital computer with data sets and instruction sets entered into a software system to practice the multiple assay methods described herein. The computer can be, e.g., a PC (Intel×86 or Pentium chip-compatible with DOS®, OS2®, WINDOWS® operating systems) a MACINTOSH®, Power PC, or SUN® work station (compatible with a LINUX or UNIX operating system) or other commercially available computer which is known to one of skill. The computer can be, e.g., a simple logic device, such as an integrated circuit or processor with memory, integrated into the system. Software for interpretation of detector signals is available, or can easily be constructed by one of skill using a standard programming language such as Visualbasic, Fortran, Basic, Java, or the like.

Multi-Assay Methods in Microfluidic Devices

Multiple analyses can be run on microfluidic devices to provide a variety of results for two or more different sample analytes and/or using two or more different assay reagents. In one aspect, a method of multiple analyses can include detecting interactions between a sample and multiple reagents to determine the presence or quantity of two or more sample analytes. In another aspect, two or more samples and two or more different reagents are received into the microfluidic device for detection of signals resulting from interactions between a matrix of samples and reagents.

As a general concept, the methods can include, e.g., completing multiple analyses by contacting a sample with two or more reagents in a microfluidic device, separately detecting signals resulting from contacts between the reagents and the sample, evaluating the detected signals for the presence or quantity of one or more sample analytes to provide-results for multiple analyses. The results can be used, e.g., to screen the sample for a variety of relevant analytes, and/or to study correlations between disease states and the multiple different assay results.

Matrices of results can be provided, e.g., by consecutively receiving two or more samples into a microfluidic device, consecutively receiving two or more different reagents into the microfluidic device, contacting the samples with the reagents in the device, and detecting signals resulting from contact of three or more different combinations of samples with reagents. Such combinations of multiple reagents and multiple samples can provide high throughput of analyses for screening and correlation studies.

Samples and Reagents of Multi-Assay Methods

Samples and reagents come into contact in microfluidic devices, e.g., to provide an array of signals and analytical results. The samples and reagents can be as described in the Multiple Assay Microfluidic Systems section, above. The samples can be, e.g., clinical samples or members of molecular libraries. The reagents can be selected, e.g., to interact with samples to generate detectable resultant signals that can be interpreted as a related set of results in screening or correlation studies.

Samples for multi-assay methods can contain one or more analytes of interest. The lack of an analyte in a sample can be detected, e.g., as lack of a signal above a validated assay sensitivity at a time a sample-associated signal would be expected at the detector. Multiple assay results can be obtained for a sample with a single analyte of interest, e.g., where two or more different reagents are sensitive to different aspects (e.g., different binding sites and/or physical parameters) of the analyte. Multiple assay results can be obtained for samples containing multiple analytes of interest. For example, assay results for the presence of an SNP, the quantity of an antigen, and a ratio of oncogenes can all be determined for a single sample on a single microfluidic device. The term “different samples”, as referred to herein, means samples that are not the same. For example, different samples can be different members of a library, samples from a different source, or samples made up of different constituents.

Reagents for multi-assay methods of the invention can include, e.g., moieties that can interact with sample analytes to provide a detectable resultant signal. The reagents are typically soluble analyte sensitive chemicals flowing in channels of a device. Such reagents often bind and/or react with the analytes to provide a resultant signal. For example the reagent can be a labeled probe that specifically binds to the analyte. The reagent can be a chromophore that reacts with the analyte to cause a changed light absorbance. In preferred embodiments, the reagent is an antibody or nucleic acid probe with a fluorescent resonant energy transfer (FRET) or molecular probe label. Some preferred reagents for the multi-assay systems include, e.g., a locus specific reagent, a PCR primer, a nucleic acid probe, a labeled ligand, a chromophore, an antibody, a fluorophore, an enzyme, a fluorescent resonant energy transfer (FRET) probe, a molecular beacon, or a radionuclide.

In other embodiments, the reagents can be, e.g., chemical groups or structures stationary in a reaction channel of a device. For example, surface bound reagents can interact with analytes to provide a resultant signal by, e.g., capturing the analyte and/or selectively resolving the analyte from other sample constituents. The capture and/or resolution can allow detection by an appropriately configured detector. Typical stationary reagents in systems of the invention include, e.g., selective media, chromatographic media, ampholytes, capture probes, affinity molecules bound to solid supports, and the like. Stationary reagents can be loaded to reaction channels in series or can be unchanging elements of a reaction channel. As used herein, “different reagents”, refers to reagents used to detect different analytes or reagents used to detect different aspects (e.g., size, light absorbance, ligand binding sites, etc.) of the same analyte. The different reagents can include combinations of reagents used in the same assay, such as, e.g., capture and label antibodies of an ELISA style assay, the enzyme labeled probe and enzyme substrate of certain chromogenic assays, the primers and probes of a PCR style assay, and the like.

In some cases, reagents of the invention can include both stationary reagents and reagents in solution or suspension. For example, a labeled antibody reagent in solution can bind to a sample analyte of interest on contact with a sample. The antibody/analyte pair can be captured by a capture antibody bound to the surface of a reaction channel/detection region for detection of a resultant signal. Unbound labeled antibody can be washed away from the capture surface so that resultant signal from reagent/sample contact can be detected in the reaction channel/detection region without high background interference.

Reagents of the invention can be a group of reagents that work together to provide a resultant signal from contact with a sample analyte of interest. For example, a PCR/FRET assay system can include contact of a sample nucleic acid analyte with two PCR primer oligonucleotide reagents, two TaqMan® probe reagents, and a thermostable DNA polymerase reagent. Polymerase chain reaction to amplify the nucleotide of interest can take place in a thermocycling amplification reaction channel before the reaction mixture flows into a fluorescence detection region.

Devices Configured for Multiple Assays

Multiple assays can be run on a single microfluidic chip by configuring reagent channels and/or sample input channels to flow one or more samples into contact with two or more reagents. Microfluidic devices can be designed, e.g., to receive two or more samples in series or parallel, to contact two or more different reagents in series or parallel, and with flows into one or more reaction channels and detection regions.

The microfluidic devices of the present invention generally comprise a body structure with microscale channels or other cavities fabricated therein. Typically, the device includes one or more reagent channels, reagent wells, sample channels, reaction channels, and/or detection regions. Samples and reagents typically flow to make contact in a microscale reaction channel of the microfluidic device before flowing into a detection region. Microchannels of a microscale device have at least one dimension (typically width and depth) ranging from about 500 μm to about 0.1 μm.

Reagent sources are typically fluidly coupled to the reaction channel. The reagent sources are typically reservoirs or wells fluidly coupled to the reaction channel for adding, removing, or storing the various reagents of interest. Alternatively, the reagent source comprises one or more pipettor tubes fluidly coupled to one or more reaction channels and configured to receive reagents from a reagent source, e.g., a microwell plate. A train of reagents is optionally stored in the microwell plate in a specimen station, which is then accessed by the pipettor tube to be received into the device.

A detection region is typically included in the devices of the present invention for the detection of signals resultant from sample/reagent interactions. The detection region is optionally a subunit of a channel, or it optionally comprises a distinct channel that is fluidly coupled to the plurality of channels in the microfluidic device, e.g., to the reaction channel. The detection region typically includes a window through which a signal is monitored. The window typically includes a transparent cover allowing visual or optical observation and detection of the resultant signals, e.g., observation of a colorimetric or fluorometric signal or label. Examples of suitable detectors are well known to those of skill in the art.

The above channels can be, e.g., fluidly coupled to each other and to various pressure sources and/or electrokinetic sources. Fluidic materials, such as samples and many reagents, are typically transported through the interconnected channel system by the application of pressure and/or electrokinetic forces to the fluid materials in the channels. Therefore, various pressure sources and electrokinetic controllers are optionally coupled to the devices of the invention. Typically, the pressure sources are applied at channel ends. For example a waste well is optionally placed at one end of a reaction channel with a sample source at the other end. A pressure source applied at the waste well is optionally used to draw fluid into the channel. For example, a vacuum source can be fluidly coupled to the device at a waste reservoir located at the efferent end of the detection region. The vacuum optionally draws any excess, or unused material into the waste reservoir to which the vacuum source is fluidly coupled. For example the vacuum pulls fluid through a porous matrix into a waste reservoir. Alternatively, a positive pressure source is fluidly coupled to a sample channel or reservoir well at one end of a reaction channel. The pressure then forces the material into and through the intended course of channels. The pressures and/or vacuum source can induce samples and reagents to flow through reaction channels for mixing and interaction (contact). Alternatively, electrokinetic forces, e.g., high or low voltages, can be applied at reservoirs to introduce materials into the channels or transport materials through the channels. For example, voltage gradients applied across a reaction channel having selective media reagents can be used to induce migration of sample analytes through the media, thus separating the analyte from other sample constituents. In other embodiments, centrifugal force can be used to flow reagents through channels.

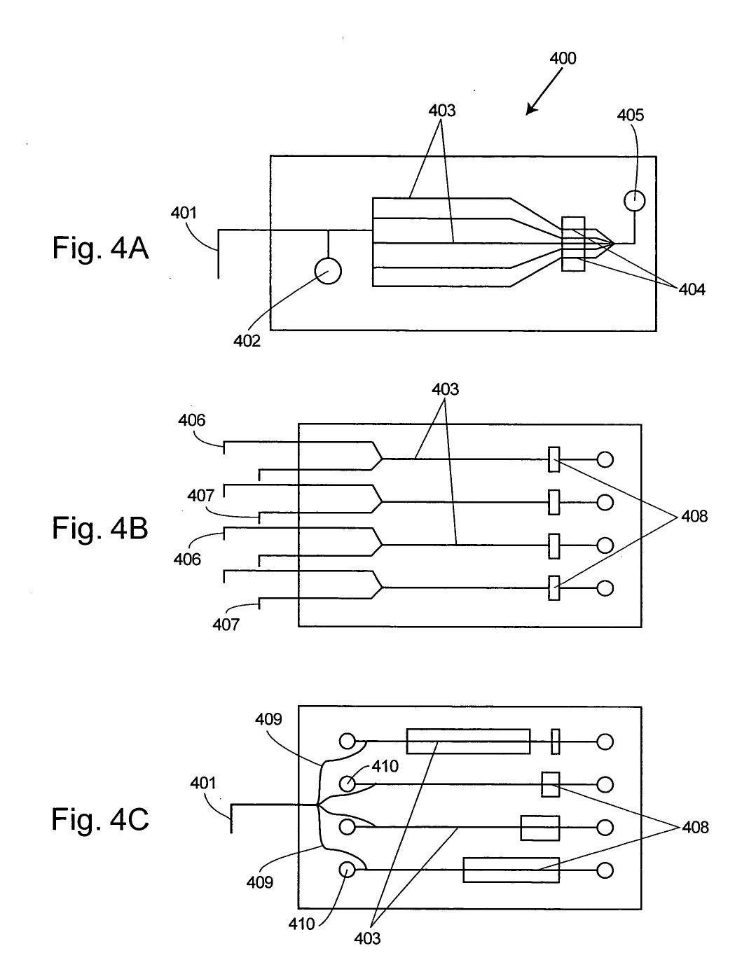

Microfluidic devices can be designed to run single or multiple samples or reagents in single or multiple reaction channels. The microfluidic devices 400 can be designed to consecutively receive a specimen from a specimen station through pipettor tube 401 and receive another specimen from well 402 on the device, as shown in FIG. 4A. The consecutive specimens can contact in multiple reaction channels 403 and be detected at multiple windows of detection region 404 before flowing to waste well 405. In another embodiment, different samples can be consecutively received in parallel through multiple sample pipettor tubes 406 while different reagents are consecutively received in parallel through reagent pipettor tubes 407 to make contact in reaction channels 403 before flowing into separate detection regions 408, as shown in FIG. 4B. Optionally, samples can be consecutively received through pipettor tube 401 to be split into separate sample channels 409 to contact different reagents from reagent wells 410 and flow through multiple reaction channels 403 that can each be configured to facilitate particular reagent/sample interactions. Resultant signals can be detected in detector regions 408 each configured for detection of particular signals from their associated assay. The systems of the invention include the various other combinations and permutations one skilled in the art can appreciate from the teachings herein.

Receiving Consecutive Samples and Reagents

The ability to consecutively receive two or more different samples and/or reagents into an analytical microfluidic device allows, e.g., high throughput screening of samples and/or development of databases large enough to provide high confidence correlations between disease states and results patterns. The small size of microfluidic devices provides fast analysis of small samples necessary for many high throughput schemes. The ability to run two or more different analyses on a single chip in methods of the invention expands benefits of microfluidics for fast and inexpensive running of “panel” assay combinations. The ability to run two or more different samples against two or more different reagents further expands the benefits to allow, e.g., massive clinical sample screening or complex correlation studies.

Analysis of consecutive samples on a microfluidic device against a single reagent is old in the art. For example a microfluidic chip can be loaded with a reagent in a reagent well and, e.g., eight different samples each in their own sample well. The microfluidic device is configured and controlled to analyze each of the samples against the reagent in turn (consecutively). Results can be obtained for each sample as it contacts the reagent and a signal is detected. The present invention adds new dimensions to this scheme by providing methods wherein more than one different analysis can be run on the same chip.

In methods of multiple analyses, samples and/or reagents can be received consecutively and/or continuously. For example, while a sample is continuously received into a microfluidic device, different reagents can be consecutively received into the device so that consecutive different analyses can be run on the same sample. Alternately, while a reagent is continuously received into a microfluidic device, different samples can be consecutively received into the device so that the same assay can be run on a set of different samples, e.g., before the reagent is changed to run a new assay on the set of samples. In a preferred embodiment, the specimen (sample or reagent) with the higher diffusivity is consecutively received while the specimen of lower diffusivity is continuously received. For example, where the sample analyte is a high molecular weight (low diffusivity) nucleic acid of interest and the reagents are lower molecular weight (higher diffusivity) oligonucleotide probes, it is preferred to intermittently sample different probes while continuously receiving the sample to provide multiple assay results. Such a plan can provide resolution and throughput benefits by lowering the effects of dispersion during consecutive analyses.

Methods of the invention can provide multiple assay results by running multiple samples and/or multiple reagents on the same microfluidic device. For example, results of two or more different analyses (based on different reagents) can be obtained for a single sample received into a microfluidic device. Optionally, e.g., a set of four different samples can received (at the same time, i.e., in parallel) to contact a set of four different reagents in separate reaction channels to provide 4 different assay results per analysis cycle. New sets of different samples and different reagents run every 6 minutes can provide 40 different assay results (40 different samples each with a different reagent). In another example, eight different samples received in parallel can consecutively contact a series of different reagents to provide, e.g., 10 consecutive analytes per hour on each of the eight samples, or 80 different assay results per hour (a matrix of 8 different samples each assayed with 10 different reagents) from the same device. In an aspect of the invention, a single microfluidic device can run in parallel more two or more different assays, 3 or more different assays, 4 or more different assays, 8 or more different assays. In methods of the invention, a single microfluidic device can run two or more different assays, 8 or more different assays, 25 or more different assays, 100 or more different assays, or 250 or more different assays, e.g., by receiving different reagents into reaction channels of the device. In methods of the invention, a sample can contact two or more different reagents, two or more different samples can each contact each of two or more different reagents, four or more different samples can each contact each of four or more different reagents, eight or more different samples can each contact each of eight or more different reagents, to provide a matrix of different assay results, e.g., including each sample/reagent combination.

Running Different Assays

Methods of microfluidic analysis in the invention can include, e.g., running two or more different assays on the same microfluidic device. Different assays use different reagents to contact samples resulting in signals associated with different sample analytes or different aspects of a sample analyte of interest. The different assays can be run consecutively and/or contemporaneously. The different assays can be run using the same reaction channel or the different assays can be run in two or more different reaction channels. In one aspect of the invention, the different assays run on the same device include one or more assays for a protein analyte and one or more assays for a nucleic acid analyte.

Different assays run on the same microfluidic device in the methods of the invention can be any type of assay known in the art. The assay can simply detect an inherent characteristic of the analyte, such as a light absorbance, fluorescent emission, molecular size, etc. The assay can include a simple chromogenic reaction. The assay can include resolution of the analyte of interest based on, e.g., charge, hydrophobicity, size, or specific affinity, using selective media reagents. The assay can include amplification reactions or schemes to enhance the signal to noise ratio. Assay interactions between analytes and reagents can take place in solution, suspension, and/or on a surface.

Different assays can be chosen to provide a combination of results useful in evaluation of a sample. For example, the different assays can be a panel of analysis useful in evaluating the condition of a medical patient. A serum electrolyte panel can include, e.g., sodium, potassium, calcium, chloride and carbonate analyses. A liver panel can include, e.g., serum bilirubin, total protein, albumin, prothrombin time, and/or enzyme levels such as alanine aminotransferase (ALT), alkaline phosphatase (ALP), aspartate aminotransferase (AST), gamma-glutamyl transferase (GGT), or lactic acid dehydrogenase (LDH). The panel of results can be reviewed by a doctor to evaluate the condition of the patient.

A combination of different assays can be useful in screening for the presence of diseases in groups of asymptomatic individuals, to diagnose the disease causing particular symptoms, or to provide prognostic information for an ill patient. For example, combinations of certain tumor markers, at certain levels (concentrations), can be indicative of the presence of particular cancers in patients. Evaluation of multiple markers and statistical correlation of combinations and levels of markers often increases confidence in the detection and identification of a cancer. Tumor markers useful for multi-assay detection and quantitation in the methods can include, e.g., alpha feto protein (AFP), human chorionic gonadotropin (HCG), beta-2 microglobulin (B2M), cancer antigens (CA 15-3, CA 19-9, CA 72-4, CA 125, and the like), carcinoembryonic antigen (CEA), prostate specific antigen (PSA), free prostate specific antigen (FPSA), FPSA/PSA ratio, neuron specific enolase (NSE), cytokeratin fragments (CYFRA), and the like. Cancerous conditions can also be associated with, e.g., high levels of plasma or serum DNA, certain levels or ratios of unfragmented DNA in clinical samples, loss of heterozygosity, microsattelite alterations, nucleic acid methylation, certain nucleic acid mutations, the presence of viral genes, and/or the like. Methods of the invention include using multi-assay microfluidic devices to analyze clinical samples for the presence and/or quantity of markers and other cancer associated analytes. The methods include statistical evaluation of data compiled from such analysis to identify, e.g., combinations of markers, and/or marker concentrations, with a high correlation to a disease state, a high sensitivity for detection of a disease state, or a low false positive error rate for detection of a disease state.

The different assays run on a microfluidic device in the methods can include, e.g., two or more assays based on the same type of reagents, and/or two or more assays based on different types of reagents. The assays can be a series of analyses, e.g., using a series of loci specific reagents (LSRs) wherein the assays are all run under essentially the same conditions but using oligonucleotide probe reagents having different sequences. Optionally, assays run on the same device can, e.g., each be based on different mechanisms of sample/reagent interaction, signal generation, and/or signal detection. For example, one reaction channel can be configured as an amplification channel to receive nucleic acid analytes and oligonucleotide reagents for fluorescence detection, another reaction channel can be an incubation channel for reaction of a ion analyte with a chromogenic reagent for absorbance detection, another channel also be a detection region including antibodies bound to a surface for immuno-capture of an antigen analyte with detection of a second labeled antibody bound to the antigen, another reaction channel can be filled with a size exclusion gel (stationary reagent) through which an analyte flows electrophoretically with detection of inherent light absorbance (e.g., at 210-260 nm), another reaction channel can incubate interaction of a molecular beacon labeled ligand binding domain with ligand analyte, and/or the like. The assays of in the multi-assay methods can include, e.g.: continuous flow PCR, flow cytometry (cells or beads), FRET, solid support binding, single molecule detection methods, electrophoresis, chromatography, mass spectroscopy, scintillation detection, and any other assay technique practicable in, or in association with, a microfluidic device.

Detecting Resultant Signals

Resultant signals in the methods of multi-assay devices can be uniform or diverse and so too can the methods of detecting the signals. Signals resulting from interaction of sample analytes with reagents can be detected, e.g., using appropriate detectors at a time or place most suitable to provide the desired detection parameters of signal intensity, sensitivity, low background, precision, accuracy, specificity, and the like. Individual detectors or combinations of detectors can be arranged to detect signals depending on the particular combinations of assays on the device.

Detectors can receive signals from detection regions of a device. The detection regions can be shared for detection of signals from different assays in turn or at the same time. The detection region is optionally a subunit of a channel, or it optionally comprises a distinct channel that is fluidly coupled to a plurality of reaction channels in the microfluidic device. Optionally, the reaction channel comprises a detection region. The detection region typically includes a window through which a signal is monitored. The window typically includes a transparent cover allowing visual or optical observation and detection of the assay results, e.g., observation of a calorimetric or fluorometric signal. Optionally, the detection region can be a channel segment with a port for sensor contact with reaction mixtures to detect certain physical or chemical signals, such as, e.g., ionic strength, pH, temperature, pressure, refractive index, capacitive conductive changes due to analyte binding to a surface, and the like. The detectors can be incorporated into the device or proximate to the device. The detectors can transmit an analog or digital detector signal (with characteristics associated with the resultant signal) thorough a communication cable to a computer for evaluation.

Interpretation and Correlation of Assay Results

Detector signals received by a computer can be interpreted to provide detection of an analyte of interest and/or quantitation of the analyte. The interpreted results for multiple assays can be used to screen samples for analytes of interest or can be correlated to draw conclusions about the status of one or more samples. The multi-assay microfluidic devices of the invention can be interpreted to provide, e.g., identification of analytes, quantitation of analytes, screening of individual samples for the presence of multiple analytes, screening of multiple samples for an analyte of interest, etc. Correlations to disease states can be drawn from presence of an analyte, the quantity of an analyte, the presence or quantity of two or more analytes, a pattern of assay results, and/or the like.

The presence of an analyte can be determined with some confidence, according assay validation skills known in the art. The presence of an analyte can be based, e.g., on detection of a signal having a predetermined magnitude above background noise for a particular assay. Failure to detect an analyte at a time expected can indicate the analyte is not present in a sample in an amount above the sensitivity of the assay. In methods of the invention, a lack of a signal can be considered a “resultant signal”.

The quantity of an analyte can be determined in many assays of the invention. The quantity of an analyte is typically related to a signal parameter, such as, e.g., a signal peak height, signal peak area, a signal shape, a signal count, and the like. Often a mathematical expression or chart of a signal parameter versus analyte concentration is prepared based on data from analysis of standard analytes of known concentration (regression analysis). The signal from an unknown concentration analyte of interest can be compared to the standard curve to determine the quantity of the analyte in a sample (with appropriate adjustments for sample dilutions, etc.).

Samples can be screened by multiple analyses on microfluidic devices of the invention to identify one or more analytes of interest. For example, a serum sample from a unit of blood can be screened for proteins and nucleic acids of interest associated with blood born diseases, such as, e.g., HCV, HIV, hepatitis A, HBV, syphilis, plasmodia, West Nile Virus, and the like. In another embodiment, food samples can be screened for pathogenic agents, such as hepatitis A, bovine spongiform encephalitis (BSE), enterobacteria, toxins, and the like. In a preferred embodiment, clinical samples can be screened to detect the presence, quantity, or pattern of tumor markers, e.g., to identify patients that might be at risk of having a cancer.

### EXAMPLES

The following examples are offered to illustrate, but not to limit the claimed invention.

### Example 1

### Screening Clinical Samples

It is understood that the examples and embodiments described herein are for illustrative purposes only and that various modifications or changes in light thereof will be suggested to persons skilled in the art and are to be included within the spirit and purview of this application and scope of the appended claims.

Clinical samples are screened for a combination of tumor markers correlated with the presence of a cancer in a patient. A multi-assay microfluidic chip is designed to include three high sensitivity assays including a RAS gene assay, a free unfragmented DNA assay, and a cancer associated antigen assay. The RAS gene assay and free DNA assay employ single molecule detection technology and the antigen assay detects antigen bound to a labeled antibody probe. The small sample size requirement, high sensitivity, fast assay cycle time, and automated sampling of the multi-assay microfluidic system allow routine screening of clinical samples for tumor markers and detection previously undiscovered cancers in a large group of patients.

Single molecule detection is based on analysis of reaction mixtures so small they often contain only one molecule of the analyte of interest, as described in copending U.S. patent application Ser. No. 10/741,162, SINGLE MOLECULE AMPLIFICATION AND DETECTION OF DNA, by Michael R. Knapp, et al., filed Dec. 19, 2003, and hereby incorporated by reference in its entirely. The sample is received to contact PCR primers and a TaqMan® probe then flow through a thermocycling reaction channel in reaction volumes so small that signals in the detection region can result from reaction of single nucleic acid target analytes with the reagents. Analyzing single molecules can have the advantage that background noise is reduced and accurate quantitation is provided by counting individual analyte molecules. Single molecule amplification techniques can be used to unambiguously determine whether a nucleotide of interest in a reaction mixture is, e.g., fragmented or has a given length between probes, as described in continuation in part application SINGLE MOLECULE AMPLIFICATION AND DETECTION OF DNA LENGTH, by Michael R. Knapp, et al., filed May 14, 2004, docket number 01-0594400US, and hereby incorporated by reference in its entirely. For example, simultaneous signals from two or more different probes hybridized to opposite ends of a nucleic acid of analyte of interest in a single copy reaction mixture provide a high level of confidence that the nucleic acid is not fragmented.

The microfluidic device of the example is designed with a single sample pipettor tube so that samples can be consecutively received into the device for the multiple different analytes. As shown in FIG. 5, consecutive samples are aliquotted from specimen station 500, drawn into sample pipettor tube 501, received into microfluidic device 502, and divided into sample channels 503 to contact reagents for the three assays.

Sample contacts the RAS PCR reagents from reagent well 504, then flows into amplification reaction channel 505 where the reaction mixture experiences 35 amplification temperature cycles before entering detection region 506. As the amplified reaction mixtures pass through the detection region, resultant signals for many single molecule reaction mixtures can be detected by detector 507. Detection signals are communicated to computer 508 for interpretation. The number of positive resultant signals in the known sample volume can be used to identify the presence of RAS and quantitate RAS nucleic acid sequences in the original sample.

Sample also contacts DNA length detection PCR reagents from well 509 then flows into amplification reaction channel 510 to be amplified in a continuous series of reaction mixtures before entering detection region 511. Over the course of the assay, resultant signals from many reaction mixtures (including single molecule and zero molecule reaction mixtures) are detected. Some single molecule reaction mixtures contain full-length DNA and the resultant signal includes two fluorescent emissions, e.g., from probes specific to sequences at each end of the full-length DNA. Other single molecule reaction mixtures contain fragmented DNA having only one end of the full-length sequence and thus provide a resultant signal of only one fluorescent emission from one probe. Resultant signals are monitored by detector 507 and transmitted to computer 508 as detector signals. The quantity and proportion of full-length and fragmented DNA can be determined by evaluation of single molecule reaction mixtures with two fluorescent emissions, single molecule reaction mixtures with one fluorescent emission, and zero molecule reaction mixtures.

The sample also contacts labeled antibodies against a cancer antigen from reagent well 512 before entering incubation region 513 of the reaction channel. After the reaction mixture has had time for antibody binding, it enters a separation region 514 of the reaction channel where unbound labeled antibody reagent is separated from antigen bound antibody. The labeled antibody:antigen complex flows into detection region 515 for detection without unbound labeled antibody background interference. Lack of a detectable signal at the time antibody:antigen is expected to enter the detection region is considered a negative resultant signal. If an amount of cancer antigen above the detectable level was present in the sample, the resultant signal indicates the presence of the antigen. Resultant signal parameters, such as peak height or peak area, can be evaluated to determine a quantity of antigen.

Proportioning and flow rates of solutions are controlled for each assay, e.g., by channel design and independent control of forces driving flows in each assay channel. The flow rate through each channel is affected by the resistance offered, e.g., by channel length, cross section, and geometry. The flow rate in each assay channel is also affected by the pressure differential between waste wells 516, reagent wells, and/or the sample pipettor tube. The flow rate is affected by electroosmotic flows induced by electric potentials across certain channels. Proper proportioning and flows of samples and reagents is thus obtained to obtain consistent and timely results for the three assays on the same device.

Detector signals associated with each consecutive sample from each of the three assays are stored in the computer for evaluation. The detector signals are evaluated for the presence of analytes of interest according to the detection of single molecule reaction mixture counts or detection of emissions of labeled antibody:antigen complex above a predetermined threshold value. The detector signals are evaluated to determine quantities of sample analytes of interest by, e.g., comparing the intensity of detector signals or the number of single molecule reactions to a standard curve including appropriate adjustments for any dilution of the sample.

The pattern of nucleic acids and antigens detected can be correlated to the presence of a cancer in the patient source of a sample. Studies using samples from large numbers of patients having known conditions (including cancer free patients and patients with known cancers) can be used to associate the presence of a marker, quantity of a marker, and/or pattern of multiple markers with certain cancers. The studies can provide marker concentrations of concern, diagnostic cancer marker concentrations, and/or marker patterns providing diagnoses or prognoses with statistically defined levels of confidence. Results for screened samples can be compared to the study data to determine whether the patient sample has marker presence, concentration, and/or pattern correlated, e.g., with the presence of a cancer.

While the foregoing invention has been described in some detail for purposes of clarity and understanding, it will be clear to one skilled in the art from a reading of this disclosure that various changes in form and detail can be made without departing from the true scope of the invention. For example, many of the techniques and apparatus described above can be used in various combinations.

All publications, patents, patent applications, and/or other documents cited in this application are incorporated by reference in their entirety for all purposes to the same extent as if each individual publication, patent, patent application, and/or other document were individually indicated to be incorporated by reference for all purposes.

What is claimed is:

1. A method of microfluidic analysis, the method comprising:
consecutively receiving two or more samples into a microfluidic device;
consecutively receiving two or more different reagents into the microfluidic device;
contacting the samples with the reagents in the device, wherein at least two of the samples contact at least one of the reagents and at least two of the reagents contact at least one of the samples; and,
detecting signals resulting from three or more of the contacts between samples and reagents.

2. The method of claim 1, wherein the two or more samples are received through a first pipettor tube and the two or more reagents are received through a second pipettor tube.

3. The method of claim 1, wherein the samples comprise one or more analytes selected from the group consisting of: a nucleic acid, a protein, a small molecule analyte, a drug, a lipid, a carbohydrate, an oncogene, a single nucleotide polymorphism (SNP), an antigen, an antibody, a methylated nucleic acid, a mutated nucleic acid, or a single molecule.

4. The method of claim 1, wherein the reagents comprise: a locus specific reagent, a PCR primer, a nucleic acid probe, a labeled ligand, a chromophore, an antibody, a fluorophore, an enzyme, a fluorescent resonant energy transfer (FRET) probe, a molecular beacon, or a radionuclide.

5. The method of claim 1, wherein two or more different samples are consecutively received into the device while one reagent is continuously received.

6. The method of claim 1, further comprising continuously receiving a higher dispersion reagent or sample while two or more of a lower dispersion reagents or samples are consecutively received.

7. The method of claim 1, wherein said contacting comprises flowing the samples or reagents in one or more microchannels.

8. The method of claim 1, wherein said contacting comprises continuous flow polymerase chain reaction (PCR).

9. The method of claim 1, wherein said detecting comprises detection of a single molecule of an analyte.

10. The method of claim 1, wherein said detecting comprises: detecting the presence of an analyte in one or more samples, detecting the absence of detectable amounts of an analyte in one or more samples, or quantifying an analyte in one or more samples.

11. A system for analyzing a matrix of sample to reagent combinations, the system comprising:
a first pipettor tube configured to sequentially receive two or more samples;
a second pipettor tube configured to sequentially receive two or more reagents;
a microfluidic device comprising a reaction channel in fluid connection with the first pipettor tube and with the second pipettor tube, whereby the samples and reagents can come into contact in the channel; and,
a detector configured to detect one or more signals resulting from the contact of one or more of the samples with one or more of the reagents;
wherein resultant signals are detected for the contact of each of two or more samples separately with each of two or more reagents, thereby analyzing a matrix of samples to reagents.

12. The system of claim 11, further comprising one or more specimen stations providing pipettor tube access to receive the samples or reagents.

13. The system of claim 12, wherein the specimen stations comprise: a carousel, a multiwell plate, or a microarray chip.

14. The system of claim 11, wherein the samples comprise sample analytes selected from the group consisting of: a nucleic acid, a protein, a small molecule analyte, a drug, a lipid, a carbohydrate, an oncogene, a single nucleotide polymorphism (SNP), an antigen, an antibody, and a single molecule.

15. The system of claim 11, wherein the reagents comprise: a locus specific reagent, a PCR primer, a nucleic acid probe, a labeled ligand, a chromophore, an antibody, a fluorophore, an enzyme, a fluorescent resonant energy transfer (FRET) probe, a molecular beacon, or a radionuclide.

16. The system of claim 11, wherein the reaction channel comprises an amplification channel.

17. The system of claim 11, wherein the microfluidic device comprises multiple reaction channels.

18. The system of claim 17, wherein the detector separately detects signals from two or more of the reaction channels in series or in parallel.

19. The system of claim 11, wherein the resultant signals comprise: a detectable signal from a reagent that has reacted with a sample analyte, the lack of a detectable signal, or a signal amplitude related to a quantity of a sample analyte.

20. The system of claim 11, wherein the detector comprises: a fluorometer, a charge coupled device, a laser, an enzyme, an enzyme substrate, a photo multiplier tube, a spectrophotometer, scanning detector, microscope, or a galvo-scanner.

21. A method of multiple analyses, the method comprising:
receiving a sample into a microfluidic device;
contacting the sample with a plurality of different reagents in the device;
separately detecting signals resulting from two or more of the contacts between the reagents and the sample; and,
evaluating the resultant signals to determine the presence or quantity of one or more sample analytes;
thereby providing multiple analysis results for the sample.

22. The method of claim 21, wherein the sample comprises: whole blood, serum, plasma, stool, urine, a vaginal secretion, cervical swab, ejaculatory fluid, synovial fluid, a biopsy, cerebrospinal fluid, or amniotic fluid.

23. The method of claim 21, wherein the one or more analytes are selected from the group consisting of: a nucleic acid, a protein, a small molecule analyte, a nucleic acid from an infective agent, a forensic nucleic acid, a drug, a lipid, a carbohydrate, an oncogene, a single nucleotide polymorphism (SNP), an antigen, an antibody, a methylated nucleic acid, a mutated nucleic acid, and a single molecule.

24. The method of claim 21, wherein the reagents comprise: a locus specific reagent, a PCR primer, a nucleic acid probe, a labeled ligand, a chromophore, an antibody, a fluorophore, an enzyme, a fluorescent resonant energy transfer (FRET) probe, a molecular beacon, or a radionuclide.

25. The method of claim 21, wherein said contacting comprises continuous flow polymerase chain reaction (PCR).

26. The method of claim 21, wherein the analyses comprise: detection of DNA mutations, detection of methylation patterns, or detection of antigens.

27. The method of claim 21, wherein the analyses comprise analysis for a nucleic acid and analysis for a peptide.

28. The method of claim 21, wherein said contacting comprises splitting the sample to flow into a plurality of reaction channels in the device to separately contact the plurality of different reagents.

29. The method of claim 21, wherein the multiple analysis results comprise screening for disease states or pathogens.

30. The method of claim 21, further comprising correlating the multiple analysis results with a disease state.

31. A system for conducting multiple sample analyses, the system comprising:
a microfluidic device comprising one or more reaction channels;
two or more different reagents in the one or more reaction channels; and,
one or more detectors configured to detect one or more signals resulting from two or more of the contacts between the reagents and one or more samples;
whereby the resultant signals can be evaluated to determine a presence or quantity of two or more analytes in the sample; thereby conducting multiple sample analyses.

32. The system of claim 31, wherein the analyses comprise: detection of DNA mutations, detection of methylation patterns, or detection of antigens.

33. The system of claim 31, wherein the one or more reaction channels comprise an amplification channel.

34. The system of claim 31, wherein the two or more reagents comprise: a locus specific reagent, a PCR primer, a nucleic acid probe, a labeled ligand, a chromophore, an antibody, a fluorophore, an enzyme, a fluorescent resonant energy transfer (FRET) probe, a molecular beacon, or a radionuclide.

35. The system of claim 31, wherein the two or more reagents contact the one or more samples in separate reaction channels.

36. The system of claim 31, wherein the sample comprises: whole blood, serum, plasma, stool, urine, a vaginal secretion, cervical swab, ejaculatory fluid, synovial fluid, a biopsy, cerebrospinal fluid, amniotic fluid, or a forensic nucleic acid.

37. The system of claim 31, wherein the two or more analytes are selected from the group consisting of: a nucleic acid, a protein, a small molecule analyte, a nucleic acid from an infective agent, a forensic nucleic acid, a drug, a lipid, a carbohydrate, an oncogene, a single nucleotide polymorphism (SNP), an antigen, an antibody, a methylated nucleic acid, a mutated nucleic acid, and a single molecule.

38. The system of claim 31, wherein the two or more analytes comprise a nucleic acid and a peptide.

39. The system of claim 31, wherein the one or more detectors comprise: a fluorometer, a charge coupled device, a laser, an enzyme, an enzyme substrate, a photo multiplier tube, a spectrophotometer, scanning detector, microscope, or a galvo-scanner.

40. The system of claim 31, further comprising a computer in functional communication with the one or more detectors to receive detector signals.

41. The system of claim 40, further comprising a system software program in the computer: to evaluate the detector signals for the presence of one or more of the analytes, evaluate the detector signals to quantify one or more of the analytes, to screen the sample for the presence of one or more pathogens, or to correlate the presence or quantity of two or more analytes to a disease state.
